(19) United States
(12) Patent Application Publication
(10) Pub. No.: US 2022/0031761 A1
(43) Pub. Date: Feb. 3, 2022

(54) COMPOSITIONS FOR PROMOTING SKIN REGENERATION, SKIN REJUVENATION, AND WOUND HEALING AND METHODS FOR PREPARATION AND USE THEREOF

(71) Applicants: Harry Thomas Temple, Miami, FL (US); Wendy Weston, Tamarac, FL (US)

(72) Inventors: Harry Thomas Temple, Miami, FL (US); Wendy Weston, Tamarac, FL (US)

(21) Appl. No.: 17/275,946

(22) PCT Filed: Sep. 11, 2019
(86) PCT No.: PCT/US2019/050500

§ 371 Date: Mar. 12, 2021

Related U.S. Application Data
(60) Provisional application No. 62/730,716, filed on Sep. 13, 2018.

(51) Int. Cl. A61K 35/50; A61K 38/18; A61K 36/886; A61K 31/4166; A61P 31/04; A61P 31/02

(52) U.S. Cl. CPC A61K 35/50; A61K 38/1833; A61P 31/02; A61K 31/4166; A61P 31/04; A61K 36/886

(57) ABSTRACT

Compositions for promoting skin regeneration, skin rejuvenation, and wound healing that include tissues derived from the human placenta, such as the amniotic membrane, chorionic membrane, and umbilical cord. The placental tissues can be combined or compounded with aloe or allantoin or both to form topical treatment creams. Additionally, these compositions have therapeutic and cosmetic uses.

### FIELD OF THE INVENTION

The invention is encompassed within the field of regenerative medicine and generally relates to compositions for promoting skin repair and regeneration, skin rejuvenation, and wound healing, particularly to such compositions including placental tissues, and most particularly to such compositions including amniotic membrane, chorion and umbilical cord tissue combined or compounded with aloe, allantoin, or both.

### BACKGROUND

Although basic principles of wound healing have been known since approximately 2000 BC, rapid advancement, particularly in products for promotion of healing, did not begin until the 20^{th} century. Modem products and techniques include wound dressings made of highly absorbent materials, allogeneic skin and dermal tissues, bioengineered tissues/skin substitutes, artificial manipulations of pressure in the wound environment (e.g. negative pressure wound therapy (NPWT) and hyperbaric oxygen therapy) (1).

Placental tissue has been described as a beneficial adjunct to wound healing that exhibits inherent anti-inflammatory (1-4), antimicrobial (3, 5-7), and epithelialization properties that are beneficial for enhancing dermal repair and skin regeneration (8-13). Placental tissues. e.g. amnion, are typically delivered as a thin sheet over an open wound or on the skin as a biologic dressing (1, 9, 12, 13, 17, 18).

Placental tissue produces secretory leukocyte proteinase inhibitor (SLPI), elafin, lactoferrin, and ß-definsins; all of which have antimicrobial actions and act as components of the immune system to provide protection from infection. In addition, lactoferrin, elafin, and SLPI all have anti-inflammatory properties. Lactoferrin is also known to suppress the production of pro-inflammatory Il-6 in the amniotic fluid during amniotic infection (3), thus mitigating the inhibition of regulatory T cells.

Both placental and umbilical tissue elute growth factors into the surrounding environment. One of these growth factors is hepatocyte growth factor (HGF). HGF is secreted by mesenchymal cells and acts primarily upon epithelial and endothelial cells. This growth factor plays a major role in embryonic development, adult organ regeneration, and wound healing (14). Since HGF has the ability to stimulate cell division and matrix invasion, it has an important role in tissue regeneration (15). Increased expression of HGF has been associated with the enhanced and scarless would healing capabilities of fibroblast cells isolated from the oral mucosa tissue (16).

Considering the immense worldwide need for effective wound-healing treatments, particularly regarding patients treated with radiotherapy and chemotherapy, burns, and chronic, non-healing wounds, treatments exhibiting increased clinical efficacy, ease of application and decreased cost are desired and necessary.

The invention described herein is intended for use in wound healing, skin regeneration, and skin rejuvenation. Potential applications include but are not limited to: skin protection and regeneration following laser surgery (e.g. tattoo removal, body sculpting, photo-rejuvenation, and laser resurfacing), healing of wounds (e.g. diabetic ulcers, wounds of chemotherapy and radiation patients), scar prevention or minimization (e.g. sutures, cosmetic surgery, and home use), and scar reduction (e.g. old scars and keloids), radiation burns, skin and dermal defects from surgery, enhanced wound healing following bone and soft tissue injury from trauma and surgery.

### SUMMARY OF THE INVENTION

The invention provides cosmetic and/or pharmaceutical compositions for rejuvenating or regenerating skin and promoting wound healing. These cosmetic and/or pharmaceutical compositions are formulated with placental tissue, particularly, but not limited to, amnion, chorion or umbilical tissue or a combination of these, combined with aloe, allantoin, or a mixture of both aloe and allantoin.

In a general aspect, the invention provides compositions having skin-rejuvenating properties.

In another general aspect, the invention provides compositions having skin-regenerating properties.

In a general aspect, the invention provides compositions having wound-healing properties.

In yet another general aspect, the invention provides compositions having scar-minimizing or scar preventive properties.

In a general aspect, the invention provides compositions having scar-reducing properties.

In another general aspect, the invention provides compositions having antimicrobial properties.

In a general aspect, the invention provides compositions for inhibition of bacterial growth.

In another general aspect, the invention provides compositions having a bacteriostatic mode of inhibition.

In yet another general aspect, the invention provides compositions having a bactericidal mode of inhibition.

In an aspect, the invention provides pharmaceutical compositions including placental tissue. The placenta is a circular organ formed in the uterus of a pregnant mammal to function as an interface between the mother and developing fetus such that the fetus is maintained and nourished. In a preferred, non-limiting embodiment, the placenta is a human placenta. The term “placental tissue” includes any tissue derived from, originating in, or associated with the placenta. A non-limiting example of placental tissue used in the described compositions is tissue derived from the amniotic membrane; i.e. the inner membrane of the amniotic sac in which the fetus develops. Another non-limiting example of placental tissue used in the described compositions is tissue derived from the chorion; i.e. the outer membrane of the amniotic sac in which the fetus develops. Another non-limiting example of placental tissue used in the described compositions is tissue derived from the umbilical cord; i.e. a conduit connecting the placenta to the developing fetus through the fetus is nourished and maintained.

In certain embodiments, the placental tissue is cryogenically micronized. Cryogenic micronization refers to a process of reducing average diameter of particles of a solid material at subzero temperatures; for example, in the described invention, micronization is a reduction of an average diameter of particles of placental tissue at liquid nitrogen temperature for inclusion in an amnion powder. Entry for “Micronization” as accessed from the Wikipedia website on Nov. 14, 2017. Placental tissue is rich with growth factors and cytokines (19-20). The use of subzero temperatures during micronization promotes the retention of these valuable growth factors within the material (23). This process enables an increase in growth factors available in the compositions to promote regeneration/rejuvenation or wound healing as compared to room temperature or simply cooled processes.

In another aspect, the invention encompasses compositions including micronized placental powder. Placental powder includes, but is not limited to, powders of placental tissues such as amniotic membrane tissue, chorionic membrane tissue and umbilical cord tissue.

In a further aspect, the invention provides a composition for promoting skin regeneration, skin rejuvenation, and wound healing composed of placental tissue combined with one or both of aloe and allantoin.

In a similar aspect, the invention provides a composition for promoting skin regeneration, skin rejuvenation, and wound healing composed of micronized placental powder combined with one or both of aloe and allantoin.

In a general aspect, the invention provides compositions containing placental tissues having both therapeutic and cosmetic applications. For example, certain embodiments provide a composition effective for promoting skin regeneration and wound healing and similar embodiments provide a composition effective for promoting or enhancing skin rejuvenation.

One aspect of the invention includes cosmetic use of the described compositions. A “cosmetic use” refers to any application of the composition for a non-therapeutic purpose. A non-limiting example of a cosmetic use is skin rejuvenation in which the composition is applied to skin (of a subject or patient) to promote restoration of a youthful appearance. The terms “subject” and “patient” refer to a human or an animal who uses or can use the inventive compositions. In certain embodiments, the subject or patient is a human.

Another aspect of the invention includes therapeutic use of the described compositions. A “therapeutic use” refers to any application of the composition for achieving a desired effect and/or reducing an undesirable effect. A non-limiting example of a therapeutic use is skin regeneration in which the composition is applied to a wound to promote skin regeneration and healing-closure of the wound. The term “wound” is intended to encompass all injuries to tissue. Non-limiting examples of wounds amenable to treatment with the inventive compositions are surgical wounds, burns, and chronic ulcers.

In another general aspect, the invention includes pharmaceutical compositions useful for skin regeneration, skin rejuvenation, and wound healing. With reference to pharmaceutical or therapeutic compositions, the phrase “effective amount” refers to the amount of a composition necessary to achieve the composition's intended function. The phrase “pharmaceutically-effective dose” refers to the amount of a composition necessary to achieve a desired pharmaceutical effect. It is often desirable to use the smallest effective dose.

The described compositions are formulated according to properties desired in the final product; e.g. a particular desired consistency, antimicrobial activity and/or content of hepatocyte growth factor (HGF). The compositions include a placental powder composed of amnion tissue, chorion tissue, umbilical cord tissue, or any combination of these, including, but not limited to 100% amnion tissue, 100% chorion tissue and 100% umbilical cord tissue. Aloe may be added to effect consistency of the final product. For example, as demonstrated in the experiments described herein, the optimal amount of aloe added ranges from about 0.75 μl to about 2 μl per cc of placenta/placental powder (FIG. 8). The term “about” in this context refers to amounts of aloe near to close to the disclosed range of 0.75 μl to about 2 μl that allows the composition to still reasonably achieve the described properties and/or function. Allantoin may be added according to ranges taken from published studies and/or added according to an FDA-approved range of 0.5% to 2.0%. For example, it is known to add allantoin to a final concentration of up to 2% (40).

The phase “therapeutically-effective amount” refers to the amount of a composition required to achieve the desired function, i.e. promotion of skin rejuvenation or skin regeneration and wound healing. The described compositions are effective in decreasing scar formation, reducing scar formation, decreasing healing time, reducing scar size or pigmentation, and decreasing incidences of complication (e.g. infection, failure to heal). Pigment differences in scars can be measured to determine effectiveness for improvements in scarring and wound sizes can be measured to determine effectiveness in wound healing. Healing time is calculated at closure of margins (of the wound). See U.S. Pat. Nos. 8,357,403; 8,372,437; and 8,409,626.

In a further aspect, the invention provides a composition in which placental tissue is combined with aloe for therapeutic enhancement, re-hydration, and cohesion of particles. Aloe is a genus of flowering succulent plants. Entry for “Aloe” as accessed from the Wikipedia website on Nov. 14, 2017. Aloe are generally ornamental plants, however, some species such as Aloe vera have pharmaceutical applications for skin and digestive discomforts (24, 25). In addition to repair of radiation damage, the immune-stimulating, anti-inflammatory, anti-microbial, and anti-neoplasm wound healing therapeutic effects of Aloe vera have been attributed to resident polysaccharides (26-28). Aloe vera has also been shown to increase the skin penetration of some compounds (29, 30). The activity of Aloe vera against gram-positive and gram-negative bacteria has been shown (31). The addition of Aloe vera to the inventive placental tissue preparation is intended to enhance the delivery of anti-microbial effects and not to impart therapeutic effect itself.

In a further aspect, the invention provides a composition in which placental tissue is combined with allantoin, also called 5-ureidohydantoin or glyoxyldiureide. Allantoin is a diureide of glyoxylic acid produced from uric acid and a major metabolite found in most organisms. Entry for “Allantoin” as accessed from the Wikipedia website on Nov. 14, 2017. Allantoin has both cosmetic applications, i.e. as an anti-aging ingredient (32), and therapeutic applications (33-35), i.e. as a treatment for a variety of skin conditions including the treatment of wounds and burns (36-39). Entry for “Allantoin” as accessed from the Natural Well Being website (Paulina Nelega) on Nov. 7, 2017.

In certain embodiments, the placental tissues are micronized and combined with at least one of aloe and allantoin to form topical treatment creams. Allantoin may be added to a final concentration of up to 2% (40). Any anti-microbial effect of the final product will not be reduced by the addition of allantoin. The final composition is usually intended to be applied as a film over the desired area. The compositions are specifically formulated as a cream or alternatively, the micronized placental tissues can be added to commercially available preparations of aloe and allantoin to form compounded, topical treatment creams.

In yet another aspect, the invention provides a method for preparing a composition for promoting skin regeneration, skin rejuvenation, and wound healing. The method includes steps of obtaining at least one of tissue from an amniotic membrane, chorion and tissue from an umbilical cord; processing the tissue and forming a powder; combining the powder with at least one of aloe and allantoin; and forming a cream composition for promoting skin regeneration and wound healing. The compositions prepared can include one or both of tissue from an amniotic membrane and tissue from an umbilical cord and one or both of aloe and allantoin. In certain embodiments, the tissue is obtained from human placenta.

An aspect of the processing step includes screening the tissue for disease; sterilizing the tissue; freeze-drying or dehydrating the tissue; and micronizing the dried tissue.

In a therapeutic aspect, the invention provides a method for promoting skin regeneration and/or wound healing in a subject in need thereof. The method includes steps of providing any of the inventive compositions described and administering the composition to the subject. The composition is usually administered to the subject as a film over the desired treatment area.

In a cosmetic aspect, the invention provides a method for promoting skin rejuvenation in a subject in need thereof. The method includes steps of providing any of the inventive compositions described and administering the composition to the subject. The composition is usually administered to the subject as a film over the desired treatment area.

In another embodiment, the invention provides a kit including the compositions of the invention.

In yet another embodiment, the invention provides a kit including ingredients for preparing the compositions of the invention. In a certain, non-limiting aspect the kit includes micronized placental powder, a cream including at least one of aloe and allantoin; and instructions for compounding the cream with the micronized placental powder.

In another aspect of the invention, any of the above-described placental powders can be used in the manufacture of any of the above-described compositions, cosmetics, and/or pharmaceutical compositions.

Other objectives and advantages of this invention will become apparent from the following description, wherein are set forth, by way of example, certain embodiments of this invention.

### BRIEF DESCRIPTION OF THE DRAWINGS

A more complete understanding of the present invention may be obtained by references to the accompanying drawings when considered in conjunction with the subsequent detailed description. The embodiments illustrated in the drawings are intended only to exemplify the invention and should not be construed as limiting the invention to the illustrated embodiments.

FIG. 1 is a micrograph showing freeze-dried placental tissue including amnion, chorion, and umbilical cord tissue.

FIGS. 2A-C are micrographs showing different views of micronized placental tissue, including amnion, chorion, and umbilical cord tissue.

FIGS. 3A-H are photographs showing different samples of micronized amnion, chorion and umbilical cord tissue. Each sample includes an optimal grinding time. This data is also shown in Table 1.

FIG. 4 is a graph showing the tissue weight prior to grinding and the grind weight after grinding. This data is for the micronized tissues shown in FIGS. 3A-H.

FIG. 5 is a graph showing % tissue loss due to the grinding process for micronized tissues shown in FIGS. 3A-H.

FIG. 6 is a graph showing Hepatocyte Growth Factor (HGF) concentration in umbilical cord tissue.

FIG. 7 is representative photographs showing zones of inhibition for gram-positive and gram-negative bacteria. These “zones of inhibition” are indicative of antibacterial activity by showing where the composition has prevented/slowed or killed bacterial growth.

FIG. 8 is a photograph showing establishment of optimal aloe concentration in the compositions. Data summarized in Table 2.

FIG. 9 shows photographs of representative placental tissue and aloe-based topical creams of various consistencies.

FIG. 10 is a graph showing average diameters of zones of inhibition of treated bacterial species against creams of varying amnion/chorion membrane AC concentrations (Table 3).

FIGS. 11A-D show results of a disk diffusion assay with Escherichia coli. FIGS. 11A-C are agar plates showing zones of inhibition obtained using several or all creams of Samples 1-8 (Table 3). FIG. 11A: Samples 1 (100%), 2(0%), positive control (30 μg tetracycline), and negative control (water). FIG. 11B: Samples 3 (40%), 4 (20%), 5 (10%), and 6 (5%). FIG. 11C: Samples 7 (3%) and 8 (1%). FIG. 11D is a bar graph showing average diameters of zone of inhibition for the assay with Escherichia coli.

FIGS. 12A-D show results of a disk diffusion assay with Staphylococcus aureus. FIGS. 12A-C are agar plates showing zones of inhibition obtained using several or all creams of Samples 1-8 (Table 3). FIG. 12A: Samples 1 (100%), 2 (0%), positive control (30 μg tetracycline), and negative control (water). FIG. 12B: Samples 3 (40%), 4 (20%), 5 (10%), and 6 (5%). FIG. 12C: Samples 7 (3%) and 8 (1%). FIG. 12D is a bar graph showing average zone of inhibition diameters for the assay with Staphylococcus aureus.

FIGS. 13A-D show results of a disk diffusion assay with Pseudomonas aeruginosa (Micrococcus pyocyaneus). FIGS. 13A-C are agar plates showing zones of inhibition obtained using several or all creams of Samples 1-8 (Table 3). FIG. 3A: Samples 1 (100%), 2 (0%), positive control (30 μg tetracycline), and negative control (water). FIG. 13B: Samples 3 (40%), 4 (20%), 5 (10%), and 6 (5%). FIG. 13C: Samples 7 (3%) and 8 (1%). FIG. 13D is a bar graph showing average zone of inhibition diameters for the assay with Pseudomonas aeruginosa.

FIGS. 14A-D show results of a disk diffusion assay with Mycobacterium smegmatis (commonly used as a surrogate model for Mycobacterium tuberculosis). FIGS. 14A-C are agar plates showing zones of inhibition obtained using several or all creams of Samples 1-8 (Table 3). FIG. 14A: Samples 1 (100%), 2 (0%), positive control (30 μg tetracycline), and negative control (water). FIG. 14B: Samples 3 (40%), 4 (20%), 5 (10%), and 6 (5%). FIG. 14C: Samples 7 (3%) and 8 (1%). FIG. 14D is a bar graph showing average diameters of zones of inhibition for the assay with Mycobacterium smegmatis.

FIGS. 15A-D show results of a disk diffusion assay with Methicillin-Resistant Staphylococcus aureus (MRSA). FIGS. 15A-C are agar plates showing zones of inhibition obtained using several or all creams of Samples 1-8 (Table 3). FIG. 15A: Samples 1 (100%), 2 (0%), positive control (30 μg tetracycline), and negative control (water). FIG. 15B: Samples 3 (40%), 4 (20%), 5 (10%), and 6 (5%). FIG. 15C: Samples 7 (3%) and 8 (1%). FIG. 15D is a bar graph showing average diameters of zones of inhibition for the assay with MRSA.

FIG. 16 shows representative experiments of the testing mode for bacterial inhibition. The agar plates exhibit variations in the modes of inhibition, bactericidal (cidal) or bacteriostatic activity (static), obtained using several or all creams of Samples 1-8 (Table 3).

FIGS. 17A-C show results of an experiment evaluating mode of bacterial inhibition, bactericidal (cidal) or bacteriostatic (static), when testing MRSA. The agar plates of FIGS. 17A-B exhibit variations in the modes of inhibition of MRSA obtained using several or all creams of Samples 1-8 (Table 3). FIG. 17C is a bar graph showing average diameters of zones of inhibition and modes of inhibition obtained when testing MRSA.

FIGS. 18A-C show results of an experiment evaluating mode of bacterial inhibition, bactericidal (cidal) or bacteriostatic (static), when testing with Mycobacterium smegmatis. The agar plates of FIGS. 18A-B exhibit variations in the modes of inhibition of with Mycobacterium smegmatis obtained using several or all creams of Samples 1-8 (Table 3). FIG. 18C is a bar graph showing average diameters of zones of inhibition and modes of inhibition obtained when testing Mycobacterium smegmatis.

FIGS. 19A-C show results of an experiment evaluating mode of bacterial inhibition, bactericidal (cidal) or bacteriostatic (static), when testing with Pseudomonas aeruginosa. The agar plates of FIGS. 19A-B exhibit variations in the modes of inhibition of with Pseudomonas aeruginosa obtained using several or all creams of Samples 1-8 (Table 3). FIG. 19C is a bar graph showing average diameters of zones of inhibition and modes of inhibition obtained when testing Pseudomonas aeruginosa.

### DETAILED DESCRIPTION OF THE INVENTION

For the purpose of promoting an understanding of the principles of the invention, reference will now be made to embodiments illustrated herein and specific language will be used to describe the same. It will nevertheless be understood that no limitation of the scope of the invention is thereby intended. Any alterations and further modification in the described compositions, powders, creams, formulations, cosmetics, methods, and kits along with any further application of the principles of the invention as described herein, are contemplated as would normally occur to one skilled in the art to which the invention relates.

The inventive compositions are both therapeutically and cosmetically applicable and can be used for promoting skin regeneration and wound healing or skin rejuvenation. The compositions include tissues derived from the placenta, such as the amniotic membrane, chorion and umbilical cord. The placental tissues are micronized and combined with at least one of aloe and allantoin to form topical treatment creams. The compositions are specifically formulated as a cream or alternatively, the micronized placental tissues can be added to commercially available preparations to form compounded, topical treatment creams.

### Placental Powder

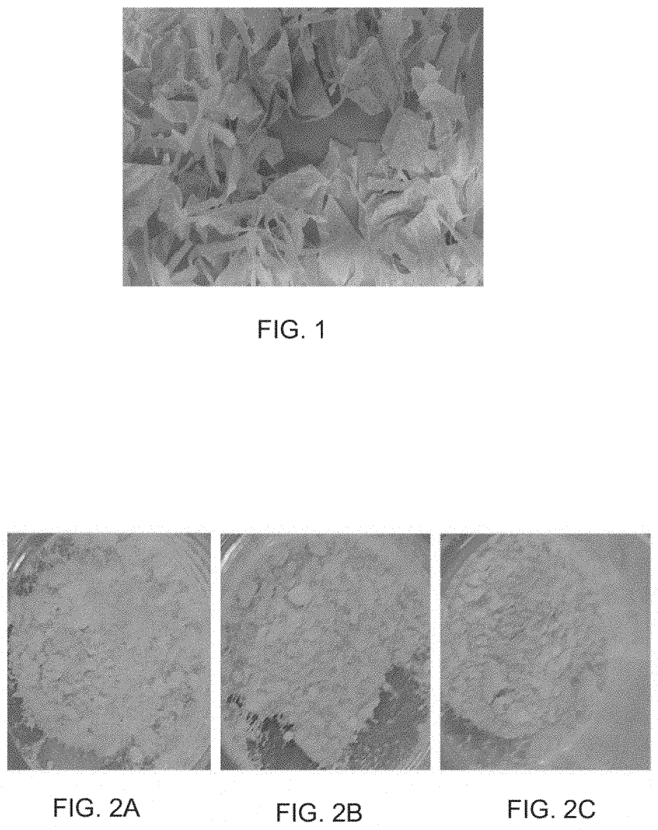

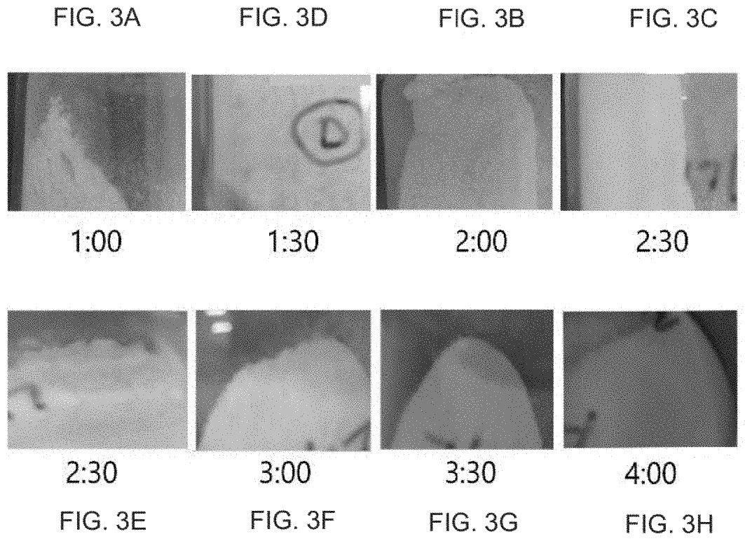

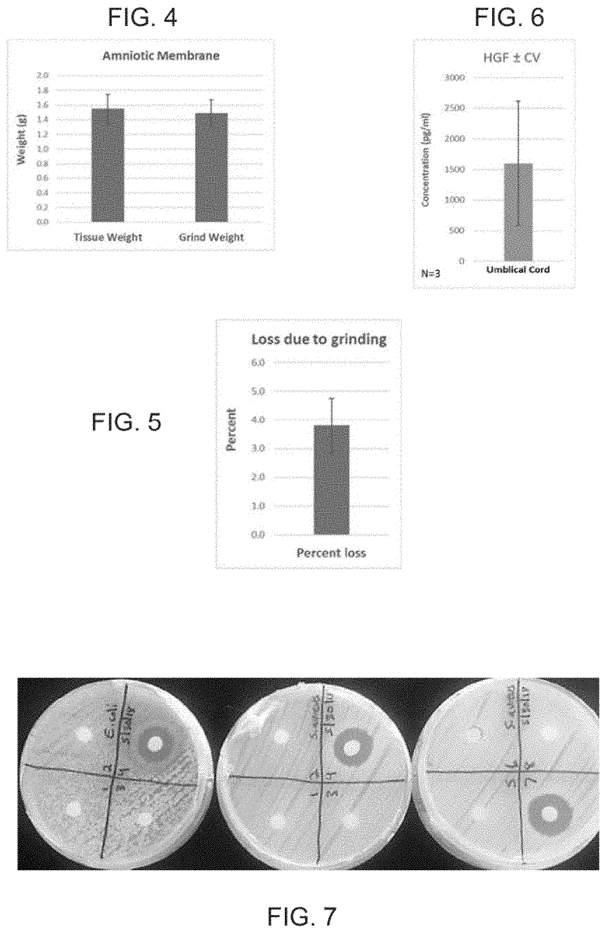

Placental powder (FIG. 1 and FIGS. 2A-C) is a primary component of the inventive composition for maximal delivery of growth factors. This component is prepared by subjecting freeze-dried or dehydrated amnion and/or chorion and/or umbilical cord (FIG. 1) for reduction of particle size, preferably, but not limited to, micronization at subzero temperatures (FIGS. 2A-C, FIGS. 3A-H, FIG. 4, and FIG. 5).

TABLE 1

| | Amnion/ | | | |
|---|---|---|---|---|
| | Chorion | UC | | Time of Run |
| Lot | (g) | (g) | Speed/sec | (min) |
| A | 0.18 | 0.54 | 30 | 2:00 |
| B | 0.18 | 0.54 | 30 | 2:30 |
| C | 0.18 | 0.54 | 30 | 3:00 |
| D | 0.18 | 0.54 | 30 | 1:00 |
| E | 0.75 | | 30 | 2:30 |
| F | 0.75 | | 30 | 3:00 |
| G | 0.75 | | 30 | 3:30 |
| H | 0.75 | | 30 | 4:00 |

Placental powders have known therapeutic efficacy in promotion of skin regeneration % rejuvenation and wound healing. The elution of HGF from the tissue over 24 hours shows that the micronized placental powder is able to locally deliver this regenerative growth factor (FIG. 6). The half-life of HGF is very short in the vascular system, approximately 3 hours. The duration of the HGF for 24 hours, at least, in the local environment points to a therapeutic advantage. In addition, the micronized particulate exhibits anti-microbial properties (FIG. 7).

### Aloe

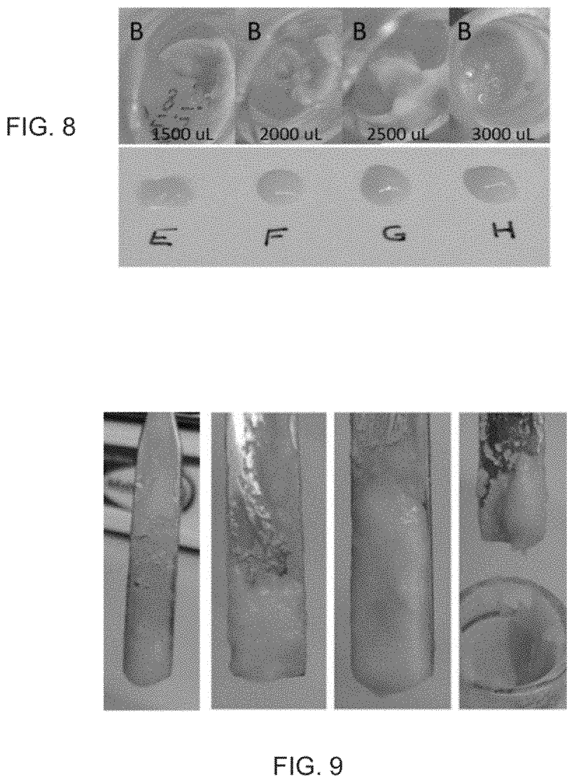

The addition of aloe to the placental tissue preparation is not intended to enhance the anti-microbial properties shown in FIG. 7, but to facilitate the delivery of anti-microbial properties of the placental tissue preparation to the tissues. For example, as demonstrated herein, the optimal amount of aloe added ranges from about 0.75 μl to about 2 μl per cc of placenta (FIG. 8). The term “about” in this context refers to amounts of aloe near to close to the disclosed range of 0.75 μl to about 2 μl that allows the composition to still reasonably achieve the described properties and/or function.

TABLE 2

| | Amount Mixed | Time of Run | Aloe Added |
|---|---|---|---|
| Lot | (cc) | (min) | (μl) |
| B | 2.0 | 2:30 | 500 |
| B | 2.0 | 2:30 | 1000 |
| B | 2.0 | 2:30 | 1500 |
| B | 2.0 | 2:30 | 2000 |
| B | 2.0 | 2:30 | 2500 |
| B | 2.0 | 2:30 | 3000 |
| E | 2.0 | 2:30 | 2250 |
| F | 2.0 | 3:00 | 2250 |
| G | 2.0 | 3:30 | 2250 |
| H | 2.0 | 4:00 | 2500 |

### Examples One-Four: Compositions

### Example One

Placental tissues, specifically amnion, chorion and/or umbilical cord are obtained from Cesarean section deliveries following informed consent of the mother. The maternal donors are screened for infectious disease with a comprehensive questionnaire (DRAI and AATB) and serology to exclude HIV, hepatitis B, hepatitis C, syphilis, and Zika virus. The processed tissues are then freeze dried or dehydrated and cryogenically ground. All donated tissues are processed aseptically and terminally sterilized using e-beam radiation. The fine powder of micronized tissue is then added to an aloe and allantoin preparation and mixed to form a cream for topical application (FIG. 9).

### Example Two

Placental tissues, specifically amnion, chorion and/or umbilical cord are obtained from Cesarean section deliveries following informed consent of the mother. The maternal donors are screened for infectious disease with a comprehensive questionnaire (DRAI and AATB) and serology to exclude HIV, hepatitis B, hepatitis C, syphilis, and Zika virus. All donated tissues are processed aseptically. The processed tissues are then freeze dried or dehydrated and cryogenically ground. The fine powder of micronized tissue is then combined with aloe and mixed to form a cream for topical application.

### Example Three

Placental tissues, specifically amnion, chorion and/or umbilical cord are obtained from Cesarean section deliveries following informed consent of the mother. The maternal donors are screened for infectious disease with a comprehensive questionnaire (DRAI and AATB) and serology to exclude HIV, hepatitis B, hepatitis C, syphilis, and Zika virus. The processed tissues are then freeze dried or dehydrated, cryogenically ground, and packaged. All donated tissues are processed aseptically and terminally sterilized using e-beam radiation. Separately, aloe or a combination of aloe and allantoin at the optimal ratio is packaged. The fine powder of micronized tissue and aloe-based diluent is provided at the optimal ratio for the desired consistency, depending on intended application. Instructions are included for the processor (e.g. nurse, preparation and distribution center) and/or the end user (e.g. nurse, physician, consumer) to prepare the final composition, which includes combination of the powder and diluent to form a cream for topical application.

### Example Four

Placental tissues, specifically amnion, chorion and/or umbilical cord are obtained from Cesarean section deliveries following informed consent of the mother. The maternal donors are screened for infectious disease with a comprehensive questionnaire (DRAI and AATB) and serology to exclude HIV, hepatitis B, hepatitis C, syphilis, and Zika virus. The processed tissues are then freeze dried or dehydrated and cryogenically ground. All donated tissues are processed aseptically and terminally sterilized using e-beam radiation. Allantoin is added at the optimal ratio and the mixture is packaged. Separately, aloe or a combination of aloe and a second diluent at the optimal ratio, is packaged. The fine powder and aloe-based diluent is provided at the optimal ratio for desired consistency, depending on intended application. Instructions are included for the processor (e.g. nurse, preparation and distribution center) and/or the end user (e.g. nurse, physician, consumer) to prepare the final composition, which includes combination of the powder and diluent to form a cream for topical application.

### Example Five: Examining Antibacterial Properties of Placental Tissue Components

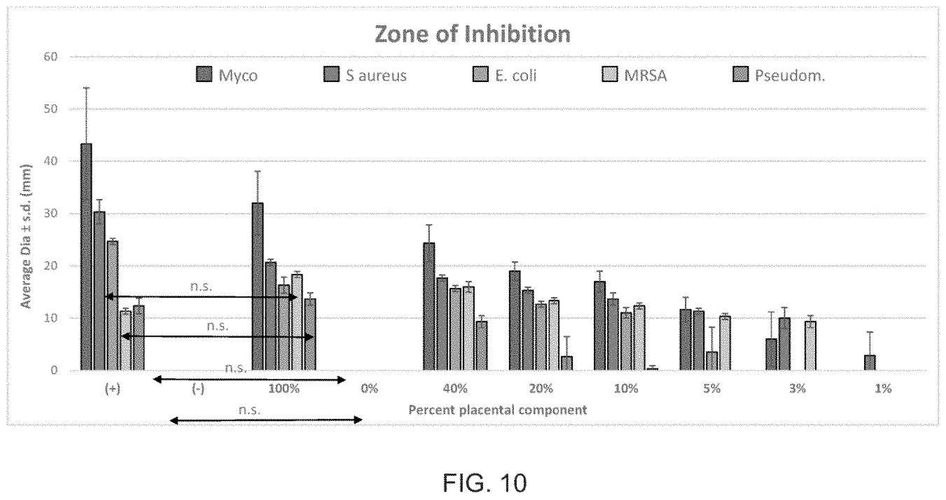

As the use of antibiotics has increased across the globe, the rate of antibiotic resistance has grown leading to a pressure to develop new antibiotics. Placental tissue is known to exhibit antimicrobial activity against bacteria common to public areas and medical facilities, making it a viable candidate for antibiotic use. Further, antimicrobial peptides have been reported to be released within placental tissue layers, for example human beta-defensin (41). However, there are no antibacterial products currently on the market that take advantage of these properties of placental tissue. With this knowledge in hand, various components of placental tissue were tested to determine the portions that exhibited antibacterial activity. Dried placental tissue samples were micronized and rehydrated. The goal of this experimental study was to take the described products and test them on multiple bacterial species, some common to human skin, found in hospital/surgical environments. The described products (creams) are contemplated for use during surgical procedures to limit chances of bacterial infection. The bacterial species tested were Escherichia coli, Staphylococcus aureus, Pseudomonas aeruginosa, Mycobacterium smegmatis, and Methicillin-Resistant Staphylococcus aureus (MRSA). A disk diffusion method was used. Bacteria were inoculated into nutrient broth, incubated overnight at 37° C., and then diluted in water to match the turbidity of a McFarland Standard. The bacteria were spread on Mueller-Hinton Agar plates. Disks with 20 uL of the hydrated tissue were placed on the inoculated plates and incubated at 37° C. overnight. Analysis of the plates showed that many of the placental tissues had a zone of inhibition around the disks, which indicated antibacterial activity (FIG. 10). The determination of whether the components exhibited bactericidal or bacteriostatic properties was seen. Bacteria within the zones of inhibition were collected, streaked onto nutrient agar plates and incubated overnight at 37° C. The analysis showed that most of the tissue components had a bacteriostatic mode of inhibition, which indicates its potential in applications intended to promote healing.

### Cream Formulations

Creams were formulated as described above through processes of micronization and rehydration. Eight samples (Table 3) were formulated having different ratios of amnion/chorion membrane (AC Placental) and umbilical cord/amnion tissue (UC:A).

TABLE 3

| | | 50:50 |
|---|---|---|
| | AC Placental | UC:A |
| Cream Sample | (%) | (%) |
| 1 | 100 | 0 |
| 2 | 0 | 100 |
| 3 | 40 | 60 |
| 4 | 20 | 80 |
| 5 | 10 | 90 |
| 6 | 5 | 95 |
| 7 | 3 | 97 |
| 8 | 1 | 99 |

### Disk Diffusion Assay

A disk diffusion assay was performed to see how the different components of the placental tissue might lead to differences in antimicrobial activity against the bacteria through analysis of the zone of inhibitions produced after the assay. Overnight cultures were made for each bacterial species and were incubated at 37° C. in a shaking incubator. The bacteria were then diluted in sterile water to match a McFarland standard and spread on MHA plates. 20 μl of each cream sample was applied on 6 mm diameter disks and aseptically placed onto the swabbed plates. A Tetracycline BBL™ Sensi-Disc™ Antimicrobial Susceptibility Test Disk was used as a positive control and distilled water was used as a negative control. 0% AC cream contained Warren Laboratories Georges Aloe Vera. The plates were incubated at 37° C. Diameters were measured after incubation.

### Results of Disk Diffusion Assay

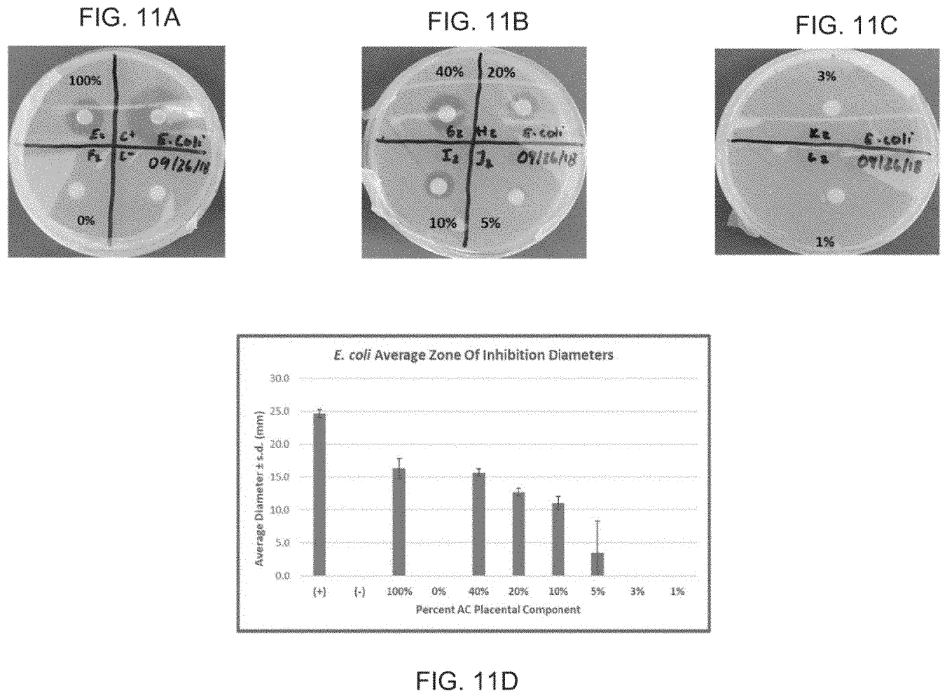

Escherichia coli(FIGS. 11A-D, Table 3)

Creams composed of more AC placental powder exhibited greater antibacterial activity (large disk diameter) and creams composed of 20% and 10% AC placental powder showed similar activity (similar disk diameters). FIG. 11A: Samples 1 (100%), 2 (0%), positive control (30 μg tetracycline), and negative control (water). FIG. 11B: Samples 3 (40%), 4 (20%), 5 (10%), and 6 (5%). Creams composed of the least amount of AC placental powder showed no antibacterial activity (no zone of inhibitions). FIG. 11C: Samples 7 (3%) and 8 (1%). FIG. 11D is a bar graph showing average diameter of zones of inhibition for the assay with Escherichia coli. Notably, there is no significant difference in the Z of I when AC component is reduced from 100% to 40%. (+) bar represents the tetracycline positive control.

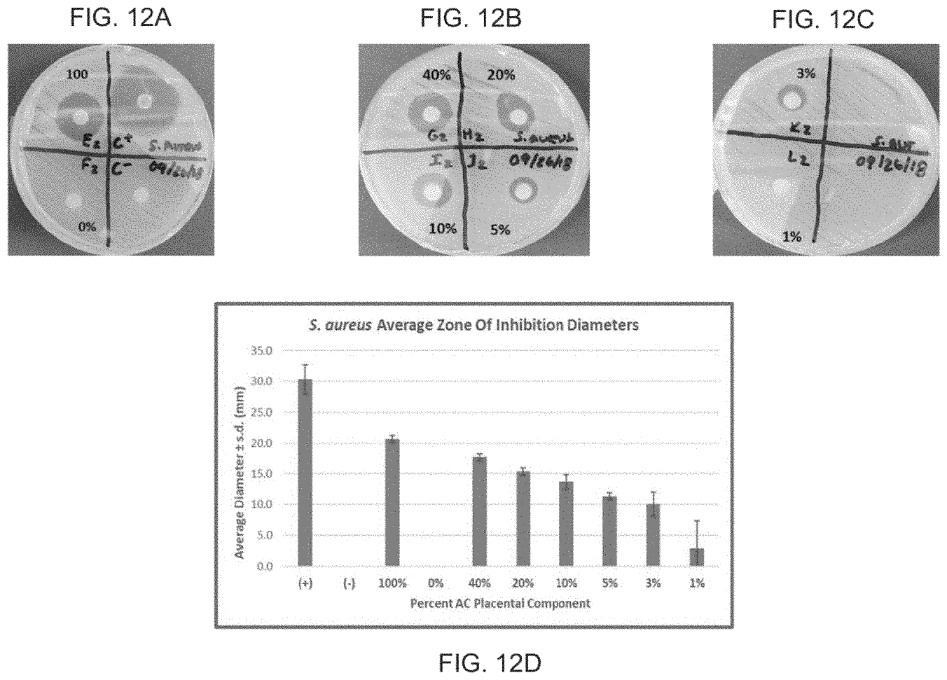

Staphylococcus aureus (FIGS. 12A-D, Table 3)

Creams composed of more AC placental powder exhibited greater antibacterial activity (large disk diameter). All creams exhibited antibacterial activity, even at the lowest concentration of 1% (Sample 8). FIG. 12A: Samples 1 (100%), 2 (0%), positive control (30 μg tetracycline), and negative control (water). FIG. 12B: Samples 3 (40%), 4 (20%), S (10%), and 6 (5%). FIG. 12C: Samples 7 (3%) and 8 (1%). FIG. 12D is a bar graph showing average diameter of zones of inhibition for the assay with Staphylococcus aureus. (+) bar represents the tetracycline positive control.

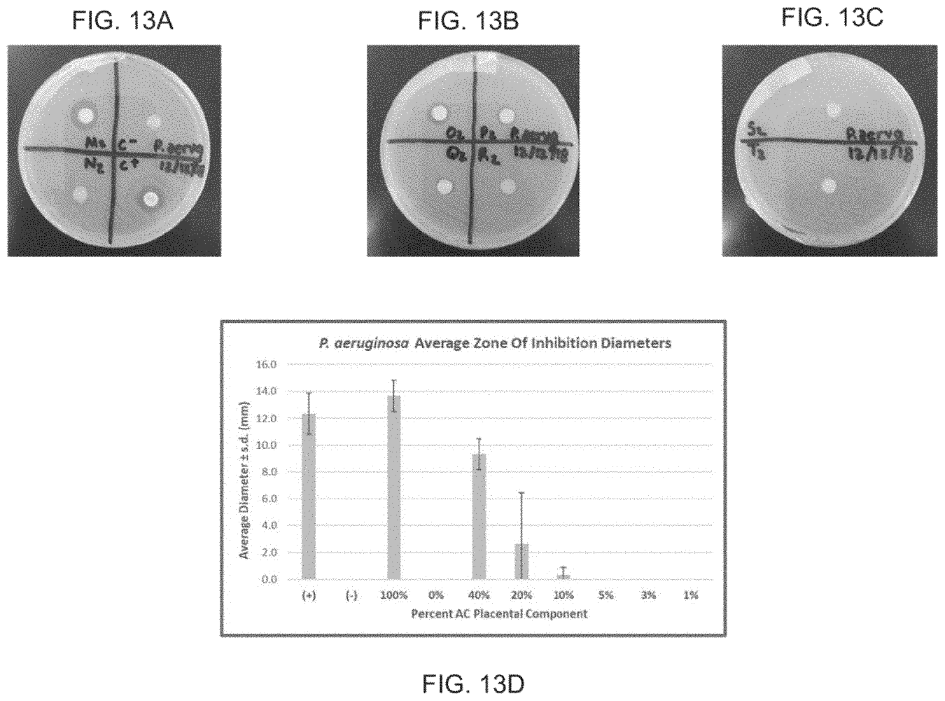

Pseudomonas aeruginosa (Micrococcus pyocyaneus) (FIGS. 13A-D, Table 3)

Creams composed of more AC placental powder exhibited greater antibacterial activity (large disk diameter). A significant drop in the zones of inhibition was observed between creams composed of 40% (Sample 3) AC placental powder and 10% (Sample 4) AC placental powder. There was no significant difference in the zones of inhibition between creams composed of 100% AC placental powder and positive control. FIG. 13A: Samples 1 (100%), 2 (0%), positive control (30 μg tetracycline), and negative control (water). FIG. 13B: Samples 3 (40%), 4 (10%), 5 (10%), and 6 (5%). FIG. 13C: Samples 7 (3%) and 8 (1%). FIG. 13D is a bar graph showing average diameters of zones of inhibition for the assay with Pseudomonas aeruginosa. (+) bar represents the tetracycline positive control.

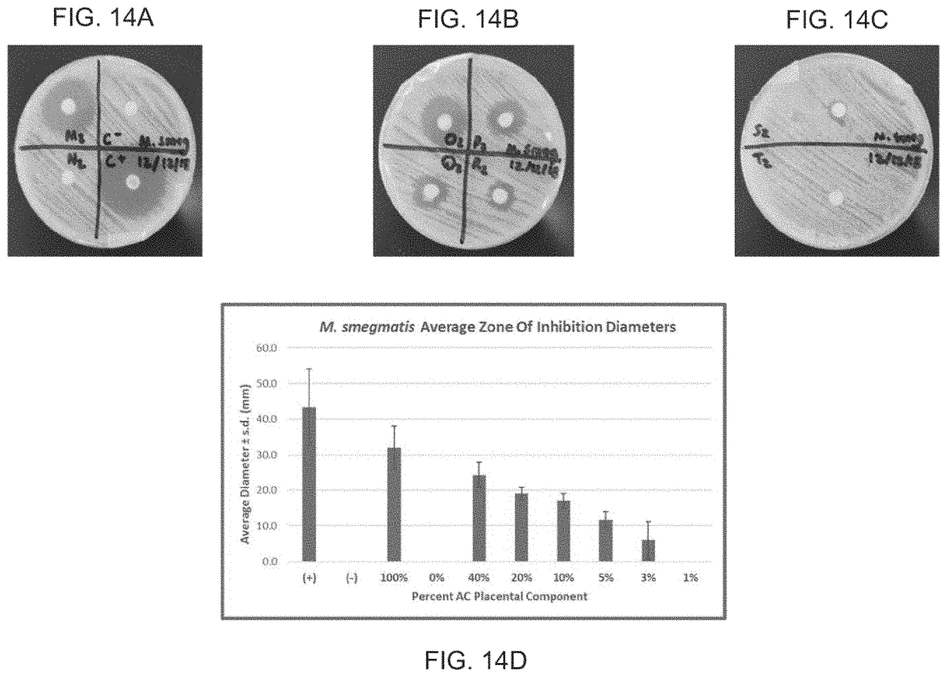

Mycobacterium smegmatis (FIGS. 14A-D, Table 3)

Creams composed of more AC placental powder exhibited greater antibacterial activity (large disk diameter). There was no significant difference in the zones of inhibition between creams composed of 100% or 40% AC placental powder and positive control. Creams composed of 1% (Sample 8) AC placental powder showed no antibacterial activity. FIG. 14A: Samples 1 (100%), 2(0%), positive control (30 μg tetracycline), and negative control (water). FIG. 14B: Samples 3 (40%), 4 (20%), 5 (10%), and 6 (5%). FIG. 14C: Samples 7 (3%) and 8 (1%). FIG. 14D is a bar graph showing average diameters of zones of inhibition for the assay with Mycobacteriun smegmatis. (+) bar represents the tetracycline positive control.

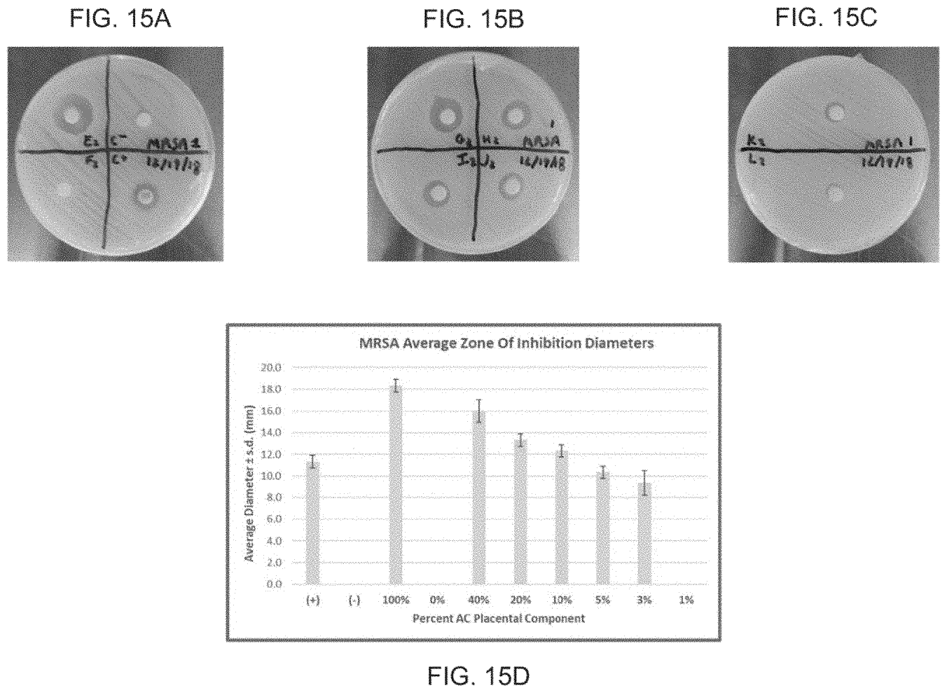

Methicillin-Resistant Staphylococcus aureus (MRSA) (FIGS. 15A-D, Table 3)

Creams composed of more AC placental powder exhibited greater antibacterial activity (large disk diameter). Creams composed of 100% (Sample 1), 40% (Sample 3), 20% (Sample 4), and 10% (Sample 5) AC placental powder had a larger zone of inhibition than the positive control with 100% (Sample 1) and 40% (Sample 3) being significantly higher. Creams composed of 1% (Sample 8) AC placental powder showed no antibacterial activity. FIG. 15A: Samples 1 (100%), 2 (0%), positive control (30 μg tetracycline), and negative control (water). FIG. 15B: Samples 3 (40%), 4 (20%), 5 (10%), and 6 (S %). FIG. 15C: Samples 7 (3%) and 8 (1%). FIG. 15D is a bar graph showing average diameters of zones of inhibition for the assay with MRSA. (+) bar represents the tetracycline positive control.

### Test for Bactericidal and Bacteriostatic Activity

The bactericidal and bacteriostatic activity was measured by swabbing the zone of inhibition on the plates followed by swabbing on fresh nutrient agar plates and incubating the newly swabbed plates overnight at 37° C. (FIG. 16).

The results showed variation in the modes of inhibition of the creams. All creams exhibited bactericidal activity at 100% and 40% concentrations.

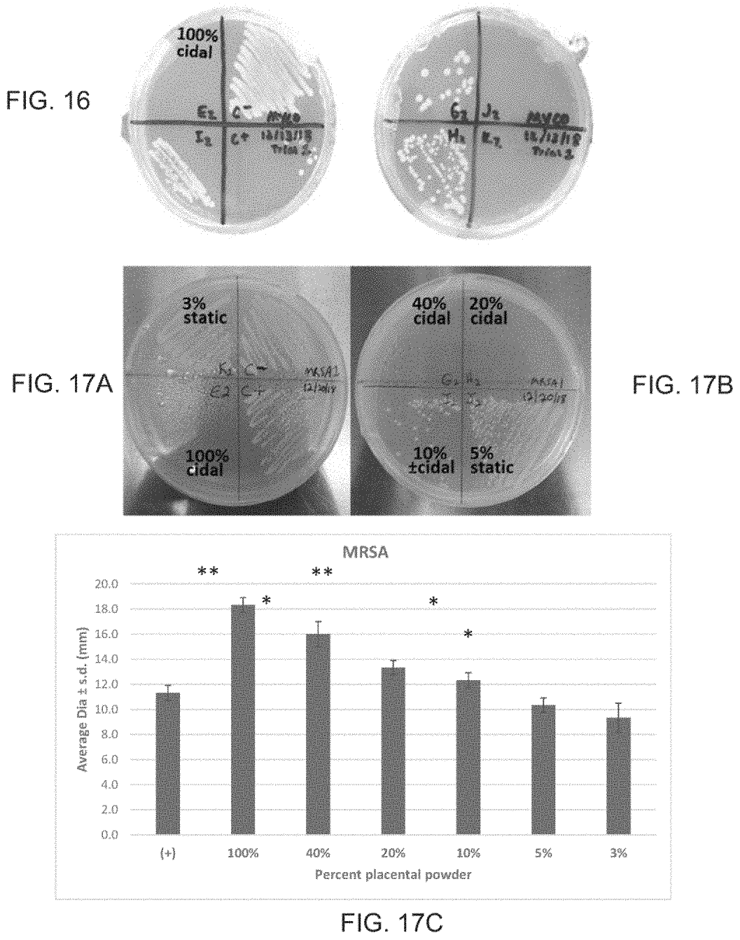

MRSA exhibited bactericidal activity at concentrations of 100%, 40%, 20%, and 10% of AC placental powder and bacteriostatic activity at concentrations of 5% and 3% AC placental powder (FIGS. 17A-C).

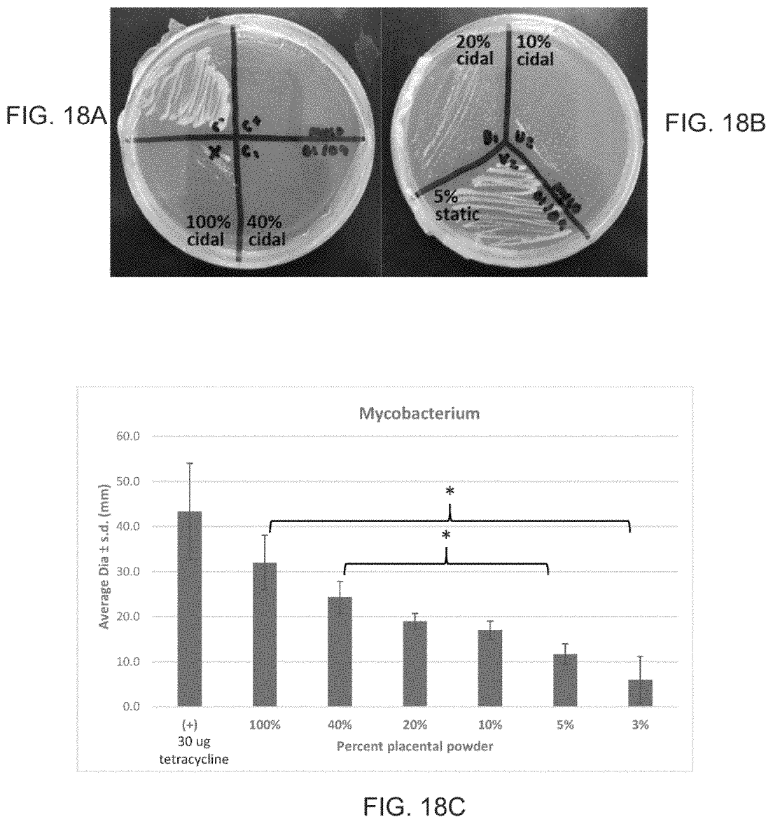

Mycobacterium smegmatis showed results similar to that of MRSA by exhibiting bactericidal activity at concentrations of 100%, 40%, 20%, and 10% of AC placental powder and bacteriostatic activity at concentrations of 5% and 3% AC placental powder (FIGS. 18A-C).

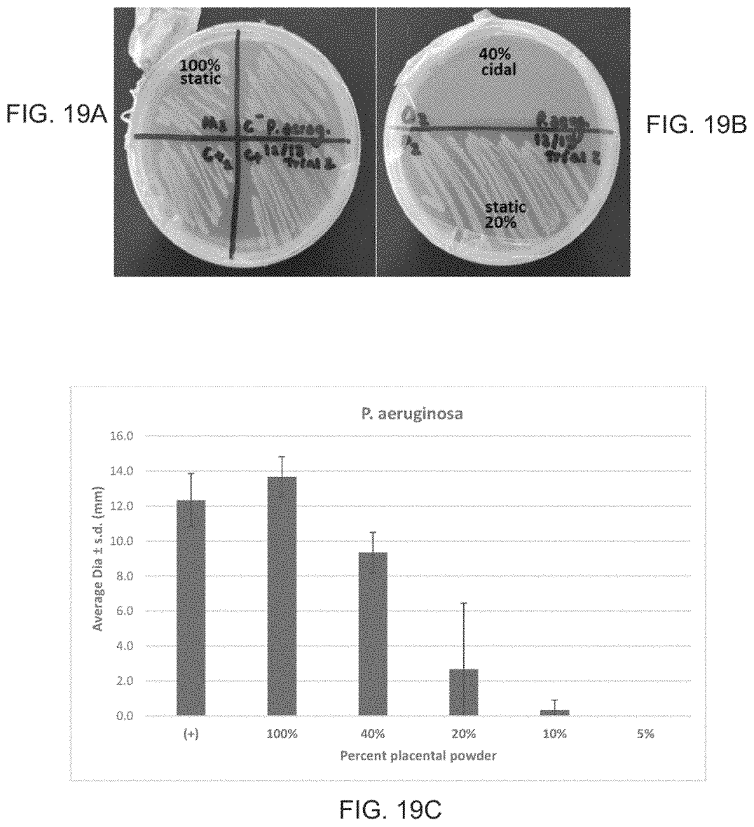

Pseudomonas aeruginosa (Micrococcus pyocyaneus) exhibited bactericidal activity at a concentration of 40% AC placental powder and bacteriostatic activity at concentrations of 100% and 20% AC placental powder (FIGS. 19A-C).

### CONCLUSION

The invention described and exemplified herein represents a new treatment composition for promoting skin regeneration or rejuvenation and wound healing. These novel compositions exhibit increased clinical efficacy and decreased costs as compared to conventional regenerative medical techniques.

All patents and publications mentioned in this specification are indicative of the levels of those skilled in the art to which the invention pertains. All patents and publications are herein incorporated by reference to the same extent as if each individual publication was specifically and individually indicated to be incorporated by reference. It is to be understood that while a certain form of the invention is illustrated, it is not intended to be limited to the specific form or arrangement herein described and shown. It will be apparent to those skilled in the art that various changes may be made without departing from the scope of the invention and the invention is not to be considered limited to what is shown and described in the specification. One skilled in the art will readily appreciate that the present invention is well adapted to carry out the objectives and obtain the ends and advantages mentioned, as well as those inherent therein. The compositions, formulations, creams, cosmetics, powders, kits, methods, procedures, and techniques described herein are presently representative of the preferred embodiments, are intended to be exemplary and are not intended as limitations on the scope. Changes therein and other uses will occur to those skilled in the art which are encompassed within the spirit of the invention. Although the invention has been described in connection with specific, preferred embodiments, it should be understood that the invention as ultimately claimed should not be unduly limited to such specific embodiments. Indeed various modifications of the described modes for carrying out the invention which are obvious to those skilled in the art are intended to be within the scope of the invention.

### REFERENCES

1. Shah S K, Jimenez F, Walker P A, Xue H, Feeley T D, Uray K S, et al. Evaluating the effects of immediate application of negative pressure therapy after decompression from abdominal compartment syndrome in an experimental porcine model. Eur J Trauma Emerg Surg. 2012; 38(1):65-73.
2. Hao Y, Ma D H, Hwang D G, Kim W S, Zhang F. Identification of antiangiogenic and antiinflammatory proteins in human amniotic membrane. Cornea. 2000; 19(3):348-52.
3. Mohan R, Bajaj A, Gundappa M. Human Amnion Membrane: Potential Applications in Oral and Periodontal Field. J Int Soc Prev Community Dent. 2017; 7(1):15-21.
4. Banas R A, Trumpower C, Bentlejewski C, Marshall V, Sing G, Zeevi A. Immunogenicity and immunomodulatory effects of amnion-derived multipotent progenitor cells. Hum Immunol. 2008; 69(6):321-8.
5. Robson M C, Krizek T J. The effect of human amniotic membranes on the bacteria population of infected rat burns. Ann Surg. 1973; 177(2):144-9.
6. Robson M C. Krizek T J, Koss N, Samburg J L. Amniotic membranes as a temporary wound dressing. Surg Gynecol Obstet. 1973; 136(6):904-6.
7. Robson M C, Samburg J L, Krizek T J. Quantitative comparison of biological dressings. J Surg Res. 1973; 14(5):431-4.
8. ElHeneidy H, Omran E, Halwagy A, Al-Inany H, AI-Ansary M, Gad A. Amniotic membrane can be a valid source for wound healing. Int J Womens Health. 2016; 8:225-31.
9. Fetterolf D E, Istwan N B, Stanziano G J. An evaluation of healing metrics associated with commonly used advanced wound care products for the treatment of chronic diabetic foot ulcers. Manag Care. 2014; 23(7):31-8.
10. Forbes J, Fetterolf D E. Dehydrated amniotic membrane allografts for the treatment of chronic wounds: a case series. J Wound Care. 2012; 21(6):290, 2, 4-6.
11. Koob T J, Rennert R, Zabek N, Massee M, Lim J J, Tenenoff J S, et al. Biological properties of dehydrated human amnion/chorion composite graft: implications for chronic wound healing. Int Wound J. 2013; 10(5):493-500.
12. Sheikh E S, Sheikh E S, Fetterolf D E. Use of dehydrated human amniotic membrane allografts to promote healing in patients with refractory non healing wounds. Int Wound J. 2014; 11(6):711-7.
13. Zelen C M, Gould L, Serena T E, Carter M J, Keller J, Li W W. A prospective, randomised, controlled, multi-centre comparative effectiveness study of healing using dehydrated human amnion/chorion membrane allograft, bioengineered skin substitute or standard of care for treatment of chronic lower extremity diabetic ulcers. Int Wound J. 2015; 12(6):724-32.
14. Gallagher J T, Lyon M. Molecular structure of Heparan Sulfate and interactions with growth factors and morphogens. In: Iozzo M, editor. Proteoglycans: structure, biology and molecular interactions. New York, N.Y.: Marcel Dekker Inc.; 2000. p. 27-59.
15. Moon P G, Lee J E, Cho Y E. Lee S J, Jung J H, Chae Y S, et al. Identification of Developmental Endothelial Locus-1 on Circulating Extracellular Vesicles as a Novel Biomarker for Early Breast Cancer Detection. Clin Cancer Res. 2016; 22(7):1757-66.
16. Dally J, Khan J S, Voisey A, Charalambous C, John H L, Woods E L, et al. Hepatocyte Growth Factor Mediates Enhanced Wound Healing Responses and Resistance to Transforming Growth Factor-beta(1)-Driven Myofibroblast Differentiation in Oral Mucosal Fibroblasts. Int J Mol Sci. 2017; 18(9).
17. Published Patent Application No. 20140342015. 2014 Nov. 20, 2014
18. Published Patent Application No. 20170203004. 2017 Jul. 20, 2017
19. Koizumi N J, Inatomi T J, Sotozono C J, Fullwood N J, Quantock A J, Kinoshita S. Growth factor mRNA and protein in preserved human amniotic membrane. Curr Eye Res. 2000; 20(3):173-7.
20. Koob T J, Lim J J, Massee M. Zabek N. Denoziere G. Properties of dehydrated human amnion/chorion composite grafts: Implications for wound repair and soft tissue regeneration. J Biomed Mater Res B Appl Biomater. 2014; 102(6):1353-62.
21. Koob T J, Lim J J, Zabek N, Massee M. Cytokines in single layer amnion allografts compared to multilayer amnion/chorion allografts for wound healing. J Biomed Mater Res B Appl Biomater. 2015; 103(5):1133-40.
22. Russo A. Bonci P, Bonci P. The effects of different preservation processes on the total protein and growth factor content in a new biological product developed from human amniotic membrane. Cell Tissue Bank. 2012; 13(2):353-61.
23. Zeisler R, Langland J K, Harrison S H. Cryogenic homogenization of biological tissues. Anal Chem. 1983; 55(14):2431-4.
24. Eshun K, He Q. Aloe vera: a valuable ingredient for the food, pharmaceutical and cosmetic industries—a review. Crit Rev Food Sci Nutr. 2004; 44(2):91-6.
25. He T P. Yan W H, Mo L E, Liang N C. Inhibitory effect of aloe-emodin on metastasis potential in HO-8910PM cell line. J Asian Nat Prod Res. 2008; 10(5-6):383-90.
26. Talmadge J, Chavez J, Jacobs L, Munger C, Chinnah T, Chow J T, et al. Fractionation of Aloe vera L. inner gel, purification and molecular profiling of activity. Int Immunopharmacol. 2004; 4(14):1757-73.
27. Ni Y, Turner D, Yates K M, Tizard I. Isolation and characterization of structural components of Aloe vera L. leaf pulp. Int Immunopharmacol. 2004; 4(14):1745-55.
28. Reynolds T. Dweck A C. Aloe vera leaf gel: a review update. J Ethnopharmacol. 1999; 68(1-3):3-37.
29. Cole L, Heard C. Skin permeation enhancement potential of Aloe Vera and a proposed mechanism of action based upon size exclusion and pull effect. Int J Pharm. 2007; 333(1-2):10-6.
30. Vinson J A, Al Kharrat H, Andreoli L. Effect of Aloe vera preparations on the human bioavailability of vitamins C and E. Phytomedicine. 2005:12(10):760-5.
31. Habeeb F, Stables G, Bradbury F, Nong S, Cameron P, Plevin R, et al. The inner gel component of Aloe vera suppresses bacterial-induced pro-inflammatory cytokines from human immune cells. Methods. 2007; 42(4):388-93.
32. Ocampo-Candiani J, Vazquez-Martinez O T, Iglesias Benavides J L, Buske K, Lehn A, Acker C. The prophylactic use of a topical scar gel containing extract of Allium cepae, allantoin, and heparin improves symptoms and appearance of cesarean-section scars compared with untreated scars. J Drugs Dermatol. 2014; 13(2):176-82.
33. Yang T T, Chiu N H, Chung H H, Hsu C T, Lee W J, Cheng J T. Stimulatory effect of allantoin on imidazoline I(1) receptors in animal and cell line. Horm Metab Res. 2012; 44(12):879-84.
34. Gus'kov EP. Prokof'ev V N, Kletskii M E, Kornienko I V, Gapurenko O A, Olekhnovich L P, et al. Allantoin as a vitamin. Doklady Biochemistry and biophysics. 2004; 398:320-4.
35. Loots J M, Loots G P, Joubert W S. The effect of allantoin on cellular multiplication in degenerating and regenerating nerves. South African medical journal=Suid-Afrikaanse tydskrif vir geneeskunde. 1979; 55(2):53-6.
36. Willital G H, Simon J. Efficacy of early initiation of a gel containing extractum cepae, heparin, and allantoin for scar treatment: an observational, noninterventional study of daily practice. J Drugs Dermatol. 2013; 12(1):38-42.
37. Araújo L U, Grabe-Guimarires A, Mosqueira V C F, Cameiro C M, Silva-Barcellos N M. Profile of wound healing process induced by allantoin. Acta Cirurgica Brasileira. 2010; 25:460-1.
38. Chan R J, Keller J, Cheuk R. Blades R, Tripcony L. Keogh S. A double-blind randomised controlled trial of a natural oil-based emulsion (Moogoo Udder Cream®) containing allantoin versus aqueous cream for managing radiation-induced skin reactions in patients with cancer. Radiation Oncology (London, England). 2012; 7:121-.
39. Fisher A A. Allantoin: a non-sensitizing topical medicament. Therapeutic effects of the addition of 5 percent allantoin to Vaseline. Cutis. 1981; 27(3):230-1, 4, 329.
40. Becker L C, Bergfcld W F, Belsito D V, Klaassen C D, Marks J G, Shank R C, et al. Final Report of the Safety Assessment of Allantoin and Its Related Complexes. International Journal of Toxicology. 2010; 29(3_suppl):84S-97S.
41. Zare-Bidaki M, Sadrinia S, Erfani S, Afkar E, Ghanbarzade N. Antimicrobial Properties of Amniotic and Chorionic Membranes: A Comparative Study of Two Human Fetal Sacs. J Reprod Infertil. 2017; 18(2):218-224.

1-47. (canceled)

48. A composition for promoting skin regeneration, skin rejuvenation, and wound healing comprising:
a micronized human placental powder comprising dried amnion and dried chorion present at an effective amount in the composition to exhibit bacteriostatic and/or bacteriocidal effects on at least one bacterium from E. coli, S. Aureus, P. aeruginosa, M. smegmatis, or Methicillin-Resistant Staphylococcus aureus (MRSA) after a twenty four hour time period of being contacted with the composition.

49. The composition of claim 48, further comprising Hepatocyte Growth Factor (HGF).

50. The composition of claim 49, wherein the composition exhibits bacteriostatic and/or bacteriocidal effects on at least two bacterium from E. coli, S. Aureus, P. aeruginosa, M. smegmatis, or Methicillin-Resistant Staphylococcus aureus (MRSA) after a twenty four hour time period of being contacted with the composition.

51. The composition of claim 50, wherein the composition exhibits bacteriostatic and/or bacteriocidal effects on at least three bacterium from E. coli, S. Aureus, P. aeruginosa, M. smegmatis, or Methicillin-Resistant Staphylococcus aureus (MRSA) after a twenty four hour time period of being contacted with the composition.

52. The composition of claim 51, wherein the composition exhibits bacteriostatic and/or bacteriocidal effects on each of E. coli, S. Aureus, P. aeruginosa, M. smegmatis, and Methicillin-Resistant Staphylococcus aureus (MRSA) after a twenty four hour time period of being contacted with the composition.

53. The composition of claim 48, wherein the composition dried amnion and dried chorion are present within the composition at an overall concentration of 20 wt % to 100 wt % of the composition.

54. The composition of claim 53, wherein the composition dried amnion and dried chorion within the composition at an overall concentration of 40 wt % to 100 wt % of the composition.

55. The composition of claim 53, further comprising at least one of aloe and allantoin.

56. The composition of claim 55, wherein the compositions comprises aloe and allantoin.

57. The composition of claim 56, wherein aloe is present in the composition at a concentration ranging from 0.75 μl to 2 μl per cc of the human placental powder and allantoin is present in the composition at an overall concentration of 0.5 wt % to 2.0 wt % of the composition.

58. The composition of claim 56, wherein the composition is configured for topical use.

59. The composition of claim 58, wherein the composition is a cream.

60. The composition of claim 52, wherein the composition dried amnion and dried chorion are present within the composition at an overall concentration of 20 wt % to 100 wt % of the composition.

61. The composition of claim 60, wherein the composition dried amnion and dried chorion within the composition at an overall concentration of 40 wt % to 100 wt % of the composition.

62. The composition of claim 61, further comprising at least one of aloe and allantoin.

63. The composition of claim 62, wherein the compositions comprises aloe and allantoin.

64. The composition of claim 63, wherein aloe is present in the composition at a concentration ranging from 0.75 μl to 2 μl per cc of the human placental powder and allantoin is present in the composition at an overall concentration of 0.5 wt % to 2.0 wt % of the composition.

65. The composition of claim 64, wherein the composition is configured for topical use.

66. The composition of claim 65, wherein the composition is a cream.
